(19) United States
(12) Patent Application Publication
(10) Pub. No.: US 2007/0059819 A1
(43) Pub. Date: Mar. 15, 2007

(54) Compost tea brewer

(75) Inventor: Stephen Storch, Water Mill, NY (US)

Correspondence Address: WILLIAM J. MASON;MACCORD MASON PLLC, POST OFFICE BOX 1489, WRIGHTSVILLE BEACH, NC, 28480, US

(73) Assignee: Progressive Gardens, LLC dba Progress Earth

(21) Appl. No.: 11/224,554

(22) Filed: Sep. 12, 2005

(51) Int. Cl. C12M 1/09

(52) U.S. Cl. 435/296.1; 435/290.1

(57) ABSTRACT

An apparatus for brewing compost tea from an aqueous compost mixture using only air bubbles as a means to circulate the mixture during fermentation. The mixture is held in a tank having a continuous sidewall and a conical bottom section with a central discharge opening. A plurality of equidistant conduits extend from the tank discharge opening to discharge nozzles having discharge ends at or just above the water level in the tank. A compressed air supply is joined by air lines to air diffusers in each of the conduits. During use, air is discharged from the diffusers to continually circulate the aqueous compost mixture upwardly in said conduits from the tank discharge opening to the discharge nozzles. The microorganisms grow rapidly in the aerated water, resulting in a rich compost tea. Due to the conical tank bottom and angled nozzles, the water swirls in the tank, creating a vortex.

### BACKGROUND OF THE INVENTION

(1) Field of the Invention

The present invention relates to an apparatus for brewing compost tea from a mixture of compost and water, and in particular to an apparatus in which small air bubbles are used to aerate the mixture is also used as the means to circulate the mixture through the brewer.

(2) Description of the Prior Art

Compost tea is a microorganism-enriched liquid extract or “tea” obtained for compost mixed with water. The tea is used commercially and by home gardeners to quickly provide nutrients to plants either through application of the tea to plants as a foliar spray, or through application of the tea to a hydroponic system or the soil around the plants.

The prior art describes many kinds of apparatus for making compost tea, ranging from a simple container in which a mixture of compost and water is held for an extended period before separation of the resultant liquid tea from the compost solids to highly mechanized devices for agitating and circulating the compost mixture.

Generally, the relatively more complex and efficient apparatus are comprised of means for agitating the mixture while introducing air into the mixture. Agitation and aeration introduction greatly speeds up the process and results in a tea that is high in aerobic microorganisms. Instead of several days, an acceptable tea with the desired microbial concentration can be produced in about 15-30 hours. The tea is then separated from the compost for use, or distributed together using an adequate spraying apparatus.

As examples of prior art systems for making compost tea, U.S. Pat. No. 6,168,949 to Rubenberger describes an apparatus in which a fermentation broth is circulating from the bottom of a tank having a conical bottom to a discharge nozzle above the upper surface of the broth in the tank where the broth is sprayed into the tank. A pump is used to convey the broth from the bottom of the tank to the spray nozzle.

U.S. Pat. No. 6,649,405 to Alms et al. describes an apparatus in which compost is placed in bags that are immersed in a tank of water. Air bubbles are then discharged from adjacent the bottom of the tank to agitate the water and extract microorganisms from the compost.

U.S. Pat. No. 6,727,090 Hronek describes an apparatus in which compost held in a first enclosure or housing is washed with water to extract micronutrients. The extract solution is then collected in a tank positioned below the housing and recirculated through the compost until the desired concentration is achieved.

U.S. Pat. No. 6,767,381 to Huhn relates to a compost tea production apparatus in which compost is placed in a basket above the water in a tank. A conduit extends within the tank from a lower end to an upper end above the basket. Compressed air is pumped into the conduit adjacent its lower end to convey water up to and out of the upper end of the conduit, where the water falls into the top of the basket.

While these prior art devices are suitable for compost tea production, there is still a need for an apparatus that will more efficiently and economically produce compost tea with a high level of microorganisms in a short period of time. The creation of a vortex as a biodynamic mechanism is also proprietary to these claims.

### SUMMARY OF THE INVENTION

The present invention relates to an apparatus in which an aqueous compost mixture is simultaneously subjected to recirculation, vortex motion and air bubble oxygenation, with air bubbles being the sole means for recirculation of the mixture.

Generally, the apparatus is comprised of a holding tank having an open top that may or may not include a cover. The tank, which will normally have a capacity of from about 50 to about 250 gallons, preferably has a conical bottom section that terminates at its lower end in a discharge opening. The tank is preferably constructed of an outdoor quality plastic, but may alternatively be constructed of stainless steel or other materials. Tanks having a circular cross-section are best suited for practice of the invention.

A plurality of conduit sections extend from the discharge opening to above the top of the tank to convey water from the bottom of the tank to the top of the tank. As used herein, the top of the tank refers to the tank at the surface of the water in the tank. Each conduit is preferably formed of first sections that extend radially outwardly from the discharge opening to distal ends beyond the circumference of the tank and second conduit sections that extend upwardly from the distal ends of the first conduit sections to upper ends near the top of the tank. The conduit radial sections may be footed to support the tank. Discharge nozzles extend inwardly from the upper ends of the second conduit sections to above the tank interior to discharge water into the top of the tank. These nozzle sections may extend through and be attached to the tank outer wall to support the tank. Preferably, the nozzles are at an angle to the nozzle axis, either clockwise or counterclockwise, depending on the hemisphere of use, to impart a swirling action to water discharged into the tank from the nozzles.

The vertical conduits, the horizontal conduits, or both, may also include means to create vortices within the conduits by forcing the water to swirl as it moves through the conduits. For example, the conduit inner walls may include rifling projections or other protrusions that are engaged by the water.

The apparatus further includes an air supply means that is connected via air lines or tubing to air diffusers within the conduits. The air diffusers may be located in either the vertical or horizontal conduits, or at the intersection of the conduits. Preferably, the air diffusers are positioned in the horizontal conduits beneath the vertical conduits. Compressed air forced through air diffusers is divided in to numerous small bubbles, greatly increasing the surface area between the air and water, and thereby greatly enhancing the oxygenation potential of the air. The preferred air diffusers are air stone, which are relatively inexpensive and are comprised of porous stones that can produce very tiny bubbles.

In operation, the tank is filled to or just below the discharge ends of the nozzles. Filling of the tank also fills the conduits to a level equal to the level of the water in the tank. Compost material in a particulate form, e.g., powdered compost, is introduced into the water and the water is stirred to disperse the compost into the water.

The air supply pump is then started, causing air to flow through the air lines and the air diffusers, resulting in a great number of small bubbles being introduced into the water above the air diffusers. These bubbles oxygenate the water and push the water upwardly and out of the nozzle discharge ends. Discharge of water from the nozzles causes circulation of water from the tank discharge opening through the conduits, exposing all of the microorganisms in the water to oxygenation by the bubbles.

In addition, due to the conical bottom section of the tank bottom, and preferably the angular orientation of the discharge nozzles, a vortex is created within the tank to further enhance mixing of the water and enhance (+) energy via resonance of the earth's spin. The extracted microorganisms, due to their repeated aeration during the water circulation, grow rapidly resulting in a desired concentration of microorganisms in the water within about 15-30 hours, e.g., about 24 hours.

Importantly, the action of the bubble is the only force causing circulation of the water. Unlike prior art devices, no pumps or other means are used to agitate the microbial laden water, potentially damaging the microorganisms and decreasing the water's ability to hold oxygen which is vital to total biomass, and thereby the suitability of the compost tea. After the desired concentration has been reached, the tea is separated from the spent compost material for use on plants.

### BRIEF DESCRIPTION OF THE DRAWINGS

FIG. 1 is a perspective view of the compost tea brewer.

FIG. 2 is a side view of the compost tea brewer.

FIG. 3 is a top view of the compost tea brewer.

FIG. 4 is a section side view of a conduit intersection showing placement of an air diffuser.

FIG. 5 is an enlarged side view of a section of vertical conduit with a section cut away to show rifling on the interior wall of the conduit.

FIG. 6 is an enlarged side view of a section of horizontal conduit with a section cut away to show protrusions on the interior of the conduit.

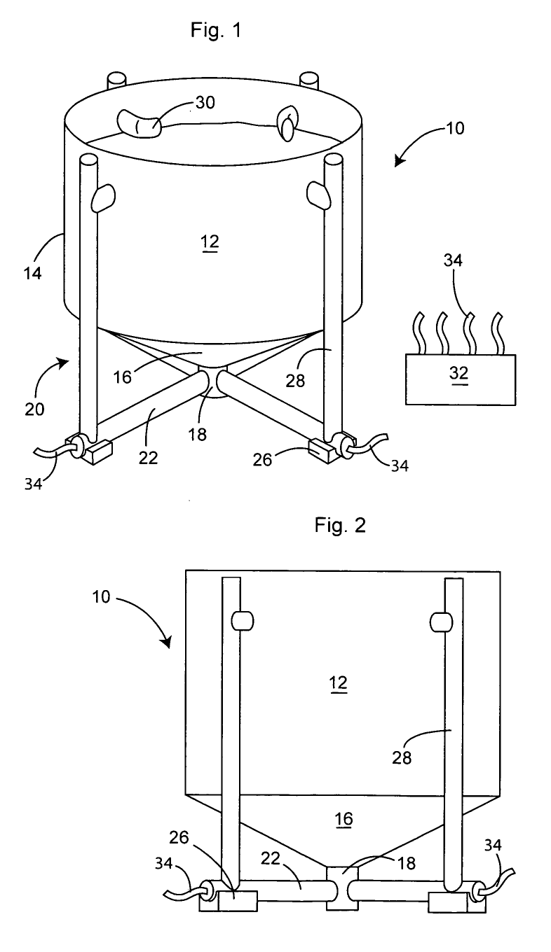

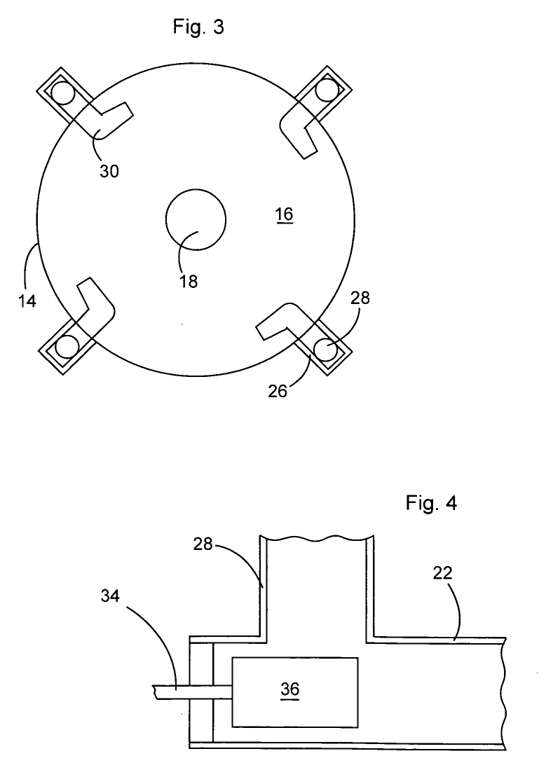

### DETAILED DESCRIPTION OF THE INVENTION

In the following description, terms such as horizontal, upright, vertical, above, below, beneath, and the like, are used solely for the purpose of clarity in illustrating the invention, and should not be taken as words of limitation. The drawings are for the purpose of illustrating the invention and are not intended to be to scale.

The compost tea brewer, generally 10, is comprised of a holding tank 12 having a continuous circular side wall 14. Tank 12 has a conical bottom section 16 terminating at its lower end in axially aligned discharge opening 18.

A plurality of conduits, generally 20, extend from discharge opening 18 to the top of tank 12. Each conduit 20 is comprised of a horizontal section 22 that extends radially outwardly from inner ends joined to discharge opening 18 to a distal end beyond the circumference of tank 12. Foot 26 forms a part of conduit section 22 to support tank 12. Each conduit 20 also includes a vertical conduit section 28 extending upwardly from the distal end of horizontal conduit section 22 to an upper end near the top of tank 12.

Discharge nozzles 30 extend inwardly from the upper ends of each of vertical conduit sections 28 to above the interior of tank 12 and, in the preferred embodiment, through side wall 14 to further support tank 12. Preferably, nozzles 30 are directed at an angle, either in a clockwise or counterclockwise direction, to enhance the swirling action to water discharged into tank 12 from nozzles 30.

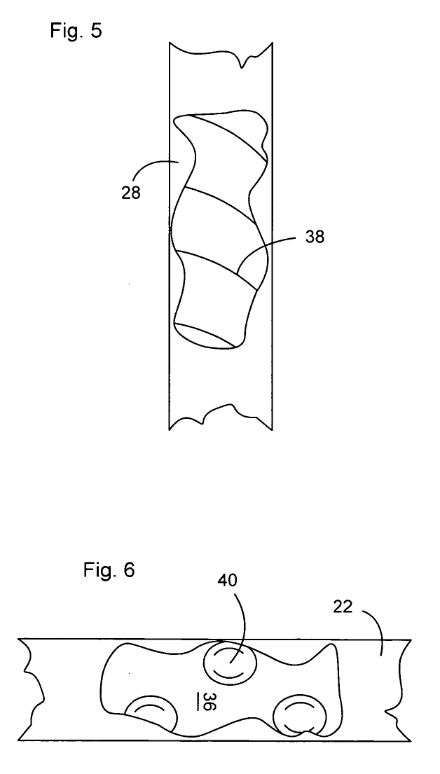

Compost tea brewer 10 further includes a compressed air supply 32, e.g., an air compressor powered by an electric motor, connected via air lines 34 to air stones 36 positioned within conduit sections 22 beneath conduit sections 28. As shown in FIGS. 5 and 6, conduit sections 22 and 28 may include rifling 38, as shown in FIG. 22, or protrusions 40, as shown in FIG. 6, or other vortex inducers to swirl the water within the conduits.

In operation, tank 12 and conduits 22 are filled with water to or just below the discharge ends of the nozzles 30. Particulate compost material is dispersed into the water within tank 12. Air supply 32 is then started, causing air to flow through air lines 34 and out of air stones 36, resulting in a great number of small bubbles being introduced into the water above air stones 36, with the bubbles moving upwardly through conduits 28 above air stones 36. Movement of the bubble pushes water upwardly and out of nozzles 30, causing water to circulate through tank 12, out of discharge opening 18 and through conduits 22, exposing all of the water and the micronutrients therein to oxygenation by the bubbles.

The flow of water past conical bottom section 16 of tank 12 enhanced by the angular orientation of nozzles 30 creates a vortex within tank 12 to disperse the compost material and separate micronutrients from the compost. After rapid growth of the extracted micronutrients in the oxygenated water to the desired concentration, e.g., in about 24 hours, the compost tea is separated from the compost and removed from tank 12 for use.

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. An apparatus for brewing compost tea from a mixture of water and compost material comprising:
a) a tank for holding the mixture of water and compost material, said tank having a continuous side wall, a top and a bottom with a discharge opening:
b) a plurality of conduits extending from said discharge opening to the top of said tank;
c) air diffusers in said conduits between said discharge opening and said tank top; and
d) an air source in communication with said air diffusers to discharge air from said air diffusers, whereby air discharged from said diffusers continually conveys water from said discharge opening to the top of said tank.

2. The apparatus of claim 1, wherein the bottom of said tank is conical, with said discharge opening being centrally located at the bottom of said conical bottom.

3. The apparatus of claim 1, wherein said conduits have angled upper discharge ends.

4. The apparatus of claim 1, wherein said tank has a circular cross-section.

5. The apparatus of claim 1, including four conduits spaced equidistant about said tank.

6. The apparatus of claim 1, wherein each of said conduits includes a first section extending outwardly from said discharge opening to a distal end beyond said tank sidewall and a second section extending upwardly from said horizontal section distal end.

7. The apparatus of claim 1, wherein said air diffusers are air stones.

8. The apparatus of claim 1, wherein said air supply communicates with said air diffusers through flexible tubing.

9. An apparatus for brewing compost tea from a mixture of water and compost material comprising:
a) a tank for holding the mixture of water and compost material, said tank having a continuous side wall and a conical bottom section with a central discharge opening:
b) a plurality of equidistant conduits extending from said discharge opening to the top of said tank, each of said conduits having an upper discharge end;
c) air diffusers in each of said conduits between said discharge opening and upper discharge end; and
d) an air compressor including flexible tubes in communication with said air diffusers to discharge air from said air diffusers, whereby air discharged from said diffusers continually coveys water upwardly in said conduits from said discharge opening to said upper discharge ends.

10. The apparatus of claim 9, wherein said conduit upper discharge ends are angled in a clockwise direction.

11. The apparatus of claim 9, wherein each of said conduits includes a horizontal section extending radially outward from said discharge opening to a distal end beyond said tank side wall, and a vertical section extending vertically upward from horizontal section distal end to an upper end, said upper end extending inwardly to above said tank.

12. The apparatus of claim 9, wherein said tank is supported by said conduits.

13. The apparatus of claim 9, wherein said conduit upper ends extend through said tank side wall to above said tank interior.

14. The apparatus of claim 9, wherein said conduit includes support feet.

15. The apparatus of claim 9, having four equidistant conduits.

16. A method of making compost tea from particulate compost material comprising:
a) forming a mixture of a particulate compost material with water in a tank having a bottom discharge opening, and conduits extending from said discharge opening to the top of said tank; and
b) continually bubbling air in said conduits while discharging said mixture in a vortex from said discharge opening and through said conduits to oxygenate micronutrients in said mixture.

17. The method of claim 16, wherein said particulate compost material is in powdered form.

18. The method of claim 16, wherein said mixture is discharged clockwise into the top of said tank.

19. The method of claim 16, wherein said tank has a conical lower section and said mixture is discharged in a vortex.

20. The method of claim 16, wherein said mixture is circulated by said air bubble for at least 15 hours.
